(19) United States
(12) Patent Application Publication
(10) Pub. No.: US 2026/0199666 A1
(43) Pub. Date: Jul. 16, 2026

(54) SOFT, STRETCHABLE AND STRAIN-INSENSITIVE BIOELECTRONICS

(71) Applicant: The Regents of the University of California, Oakland, CA (US)

(72) Inventors: Sam EMAMINEJAD, Los Angeles, CA (US); Qibing PEI, Los Angeles, CA (US); Bo WANG, Los Angeles, CA (US); Yichao ZHAO, Los Angeles, CA (US)

(73) Assignee: The Regents of the University of California, Oakland, CA (US)

(21) Appl. No.: 19/136,816

(22) PCT Filed: Dec. 7, 2023
(86) PCT No.: PCT/US2023/082932

§ 371 Date: Jun. 6, 2025

Related U.S. Application Data
(60) Provisional application No. 63/431,302, filed on Dec. 8, 2022.

(51) Int. Cl. A61N 1/05; H01B 1/02; H01B 1/08; H01B 1/22

(52) U.S. Cl. CPC A61N 1/05; H01B 1/02; H01B 1/08; H01B 1/22

(57) ABSTRACT

Advancing electronics to interact with tissue necessitates meeting material constraints in electrochemical, electrical, and mechanical domains, simultaneously. Clinical bioelectrodes with established electrochemical functionalities are rigid and mechanically-mismatched with tissue. While conductive materials with tissue-like softness/stretchability are demonstrated, when applied to electrochemically probe tissue, their performance is distorted by strain and corrosion. Here, presented is a layered-architectural composite design, which couples strain-induced cracked films with a strain-isolated out-of-plane conductive pathway and in-plane nanowire networks, to eliminate strain effects on the device electrochemical performance. Accordingly, a library of stretchable, highly conductive, and strain-insensitive bioelectrodes is provided featuring clinically-established brittle interfacial materials (iridium-oxide, gold, platinum, carbon). These bioelectrodes can be paired with different electrochemical probing methods (amperometry, voltammetry, potentiometry), thereby demonstrating strain-insensitive sensing of multiple biomarkers and in-vivo neuromodulation.

### CROSS-REFERENCE TO RELATED APPLICATIONS

This application claims priority to U.S. Provisional Patent Application No. 63/431,302 filed Dec. 8, 2022, the contents of which are incorporated herein by reference in their entirety.

### STATEMENT OF GOVERNMENT SPONSORED RESEARCH

This invention was made with government support under DK128711 awarded by the National Institutes of Health, and 1847729 awarded by the National Science Foundation. The government has certain rights in the invention.

### TECHNICAL FIELD

The present embodiments relate generally to bioelectronics, and more particularly to a new library of soft, stretchable, strain-insensitive bioelectronics featuring materials such as, but not limited to, brittle interfacial materials.

### BACKGROUND

Interfacing electronics with biological tissues is the foundation of probing and actuating biological systems. Reliable interaction between biological tissues and electronics entails materials that support underlying electrochemical and electrical processes, while satisfying tissue-imposed mechanical constraints. The electrochemical process involves electron transfer and accumulation at the bioelectronics-tissue interface for in-situ stimulation (e.g., neuromodulation) and sensing (e.g., biomarker molecules), necessitating the use of suitable interfacial materials. The electrical process relates to electron transport for signal routing and setting intended operating points (e.g., voltage), requiring the use of highly conductive materials. From the mechanics standpoint, materials that mimic tissue properties such as stretchability (~20%-75%) and compliance (Young's modulus E below or in the kPa to MPa range) are needed to establish conformable contact with tissue for high fidelity sensing/stimulation. This requirement is important for minimizing the tissue function disruption as well as the risk of implant complications resulting from scar formation, lead fracture, and tissue injury. However, the electrochemical and electrical performance of these materials may be affected by the strain originating from the movement and complex topography of the tissue.

Clinically established interfacial materials with high electrochemical performance for bioelectronics-tissue interfaces (e.g., noble metal-based ones) are all rigid (E>1 GPa) and brittle (fracture strain <1%). Recent advances in soft and stretchable conductors have enabled the development of strain-resilient devices with tissue-like mechanical properties, which maintain their electrical connection despite being extensively stretched (~800%). While these strain-resilient devices are suitable for construction and integration of soft, stretchable, solid-state electronics, when they are applied to electrochemically probe the tissue environment (involving ionic biofluid surrounding), their performance is distorted by strain- or corrosion-related issues. The former issue is manifested in devices with relatively low intrinsic conductivity (e.g., graphene, PEDOT:PSS: 1×10³ S/m), wherein strain-induced changes to their resistance cause significant deviation from the intended operating points (e.g., activation voltage), subsequently corrupting the reactions at the interface. The latter issue is especially observed in devices with highly conductive materials that are rapidly oxidized upon exposure to the surrounding solution (e.g., silver-based nanomaterials), despite applying surface modification (prone to surface defects/pores). As such, this group of devices cannot sustain the wide voltage range required to perform electrochemical reactions.

It is against this technological backdrop that the present Applicant sought a technological solution to these and other problems rooted in this technology.

### SUMMARY

The present embodiments relate generally to a new library of soft, stretchable, strain-insensitive bioelectronics featuring brittle interfacial materials. One or more embodiments relate to a layered-architectural composite design that centers on decoupling the bioelectronics materials configuration into an interfacial element for electron transfer and an interconnection element for electron transport. This design allows for exploiting and coupling surface channel cracks (within the brittle interfacial element) and anisotropic out-of-plane/in-plane electron conduction (within the interconnection element) to eliminate strain effects on the device performance. It further allows for a broad selection for the interfacial materials (including brittle noble metal-based materials). Following this approach, created was a functionally diverse library of thin (~140 μm), soft (~10 MPa, FIG. 5), stretchable (>150%), highly conductive (~1 Ω/sq) and strain-insensitive bioelectrodes (SIBs). It should be noted that the presented design principle is generalizable to a diverse range of materials; nevertheless, for ease of illustration, its utility with brittle materials described in detail herein shows an example of an extreme (difficult) case.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIGS. 1A to 1F illustrate example aspects of soft strain-insensitive bioelectrode (SIB)—such as architecture, strain dissipation mechanisms and applications—according to embodiments.

FIGS. 2A to 2G provide example visualization and electrochemical characterization of example Au-SIBs performance under strain according to embodiments.

FIGS. 3A to 3E illustrate aspects of an example library of SIBs featuring brittle interfacial materials for electrochemical sensing and stimulation according to embodiments.

FIGS. 4A to 4K illustrate example aspects of In-vivo neuromodulation via stimulation of the sciatic nerve by IrOx-SIB under deformation according to embodiments.

FIGS. 5A and B illustrate example mechanical characterizations of the ACF/AgNW/PUA composite of embodiments: (A) Uniaxial stress-strain curve of the ACF/AgNW/PUA. The ACF/AgNW/PUA can be stretched over 150%. Inset shows the tested device under tension. (B) Dynamic modulus characteristic of the composite.

FIGS. 6A to 6F provide microscopic images of strain-accommodating features of SIB within each layer according to embodiments: Provided are SEM images of (A) cracked fragments under strain (top layer; interfacial material: Au), (B) embedded conductive elements within ACF (middle layer), and (C) AgNW network (bottom layer). (D) Zoom-in view of the AgNW layer illustrating submicron features. (E, F) Cross-sectional optical microscopic images of SIB illustrating the ACF and PUA layers under outer (E) and inner bending (F).

FIG. 7 illustrates example sheet resistance of embodiments under strain. Comparison of the sheet resistance of the devised SIB (here, Au as the interfacial layer) and standalone AgNW/PUA vs. other stretchable conductors/electrodes, over the strain range of 0 to 100% (N=3; error bars indicate SEM).

FIGS. 8A to 8C provide SEM images of example electrochemically-stained electrodes according to embodiments under strain. These provide corresponding original SEM images of the electrochemically-stained electrodes illustrated in FIG. 2.

FIGS. 9A and 9B illustrate example SIB's cracked fragment size distribution under strain: (A) Characterization of the crack spacing of the SIB's interfacial layer (here, Au) at 20%, 40% and 80% strains using SEM imaging. The results indicate that the great majority of the cracked fragments' sizes are >100 μm across all strains. (B) Estimated percentage of cumulative connected cracked fragments with respect to the original (uncracked) interfacial area (based on the analyzed SEM images of the cracked fragments' area and the statistical model described below).

FIGS. 10A to 10C illustrate example SIB durability under cyclic stretching according to embodiments: (A) Characterization of the crack density of SIB's interfacial layer (here, Au) after 1 and 10,000 cycle(s) using SEM imaging (strain: 20%). Representative images of the formed cracks are shown atop. (B) SIB's resistance (red: at 20% strain; blue: at 0% strain) as a function of stretching cycles (test device: Au-SIB; strain: 20%; stretching rate: 12 mm/min). (C) Zoom-in view of real-time device resistance measurements within the time period in between the ~9000th and 9020th cycles. The measurements indicate minimal change in device resistance (~2Ω) under dynamic stretching strain.

FIG. 11 illustrates example evaluation of EIS strain-insensitivity across different Au-SIBs according to embodiments. EIS measurements of three devices illustrate their strain insensitive performance. The p values comparing unrestrained vs. strained measurements for all devices are within the range of 0.12-0.98 (strain: 100%). Error bars indicate SEM. N=3.

FIG. 12 illustrates example EIS characterization of Au-SIB under mechanical cycling test according to embodiments. Initial and corresponding impedance spectrum of Au-SIB after 100, 200, 400, 800, and 1000 cycles of 10% uniaxial tensile strain, illustrating minimal impedance magnitude variation (<8%, across the tested frequency range, after 1000 cycles).

FIGS. 13A to 13C illustrate example effects of the interconnect resistance (Ric) increase on the EIS and CV curves of non-strain resilient Au/PUA electrodes according to embodiments: (A) Impedance spectrum of a non-strain resilient model electrode (Au/PUA) with different interconnect resistances. Ric is changed by connecting different loading resistors (120, 1.5 k, 15 k ohm) in series with the interconnection trace. (B) CV profiles of the Au/PUA electrode with different interconnect resistances. (C) Comparison of the CV profile of the unstrained vs. under-strain Au/PUA, illustrating that the electrode becomes open circuit under the applied strain.

FIGS. 14A and 14B illustrate example electrochemical stability of electrodes according to embodiments. CV plots corresponding to the 1st and 50th cycle of voltammetry (0.1 M Na₂SO₄ aqueous solution), performed with a bare AgNW/PUA electrode (A) and Au-SIB (B).

FIG. 15 illustrates example progressive CV characterization of Au/AgNW/PUA electrode (post-strained) according to embodiments. The marked peaks are indicative of the unwanted silver oxidation and reduction processes, which occur due to the crack-induced exposure of the AgNWs to the electrolyte. The inset SEM figure shows the cracked Au-film and the exposed AgNWs (oxidized) underneath.

FIGS. 16A to 16C provide example evaluation of device-to-device electrochemical performance variation according to embodiments: (A) Pt-SIB's responses to 0, 50, 100 μM H₂O₂ concentration. (B) Carbon-SIB's responses to 0-50 μM APAP concentration. (C) IrOx-SIB's responses to pH conditions from 6.5 to 8. N=3 across all measurements and error bars indicate SEM.

FIGS. 17A to 17C illustrate example repeatability of SIB's strain-insensitive electrochemical performance according to embodiments: (A) Pt-SIB's H₂O₂ responses (0, 50, 100 μM) at 40% tensile strain vs. unstrained. (B) Carbon-SIB's APAP responses at 100% tensile strain vs. unstrained. (C) IrOx-SIB's pH OCP responses at 40% tensile strain vs. unstrained. N=3 across all measurements and error bars indicate SEM.

FIGS. 18A to 18C illustrate example electrochemical sensing responses of Pt- and IrOx-SIBs under dynamic tensile strain according to embodiments: (A) Amperometric current profiles of a Pt-SIB in PBS solution under 20% dynamic tensile strain. It illustrates that the intermittently established steady-state baseline current stays the same, despite inevitable transient double layer capacitance discharging (INF in FIG. 3C (ii)), which manifests as a current pulse (during stress loading/unloading). (B) Temporal pH responses of an IrOx-SIB under 20% dynamic tensile strain. (C) Ex-vivo stimulation current profile of an IrOx-SIB under 20% dynamic tensile strain (upon application of biphasic voltage pulses).

FIG. 19 illustrates example strain-insensitive H₂O₂-sensing using Au as an interfacial material. Comparison of device responses to H₂O₂ (0-6 mM; −0.25 V) at 20% tensile strain vs. unstrained. N=6 across all measurements and error bars indicate SEM.

FIG. 20 illustrates example CSC characterization of IrOx-SIB and ACF/AgNW/PUA electrodes according to embodiments. CV profiles of an IrOx-SIB and a bare ACF/AgNW/PUA, illustrating the IrOx-SIB's high CSC (11.7 mC/cm2) and the electrochemical inertness of the bare ACF/AgNW/PUA electrode.

FIG. 21 illustrates example evaluation of EIS strain-insensitivity across different IrOx-SIBs according to embodiments. EIS measurements of three devices illustrate their strain insensitive performance. The p values comparing unrestrained vs. strained measurements for all devices are within the range of 0.50-0.99 (strain: 40%). Error bars indicate SEM. N=3.

FIG. 22 illustrates an example ex-vivo biocompatibility test of embodiments. The results are from an example cellular viability test. The cell toxicity was evaluated based on the mouse embryonic fibroblasts system. NC: negative control (blank). PC: positive control (Ag ion-spiked). Error bars indicate SD.

### DETAILED DESCRIPTION

The present embodiments will now be described in detail with reference to the drawings, which are provided as illustrative examples of the embodiments so as to enable those skilled in the art to practice the embodiments and alternatives apparent to those skilled in the art. Notably, the figures and examples below are not meant to limit the scope of the present embodiments to a single embodiment, but other embodiments are possible by way of interchange of some or all of the described or illustrated elements. Moreover, where certain elements of the present embodiments can be partially or fully implemented using known components, only those portions of such known components that are necessary for an understanding of the present embodiments will be described, and detailed descriptions of other portions of such known components will be omitted so as not to obscure the present embodiments. Embodiments described as being implemented in software should not be limited thereto, but can include embodiments implemented in hardware, or combinations of software and hardware, and vice-versa, as will be apparent to those skilled in the art, unless otherwise specified herein. In the present specification, an embodiment showing a singular component should not be considered limiting; rather, the present disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present embodiments encompass present and future known equivalents to the known components referred to herein by way of illustration.

The present embodiments relate generally to a new library of soft, stretchable, strain-insensitive bioelectronics, for example those featuring brittle interfacial materials.

As set forth above, interfacing electronics with biological tissues is the foundation of probing and actuating biological systems. Reliable interaction between biological tissues and electronics entails materials that support underlying electrochemical and electrical processes, while satisfying tissue-imposed mechanical constraints (R. Feiner, T. Dvir, Tissue-electronics interfaces: from implantable devices to engineered tissues. Nat. Rev. Mater. 3, (2018). doi: 10.1038/natrevmats.2017.76; S. P. Lacour, G. Courtine, J. Guck, Materials and technologies for soft implantable neuroprostheses. Nat. Rev. Mater. 1, (2016) doi: 10.1038/natrevmats.2016.63). The electrochemical process involves electron transfer and accumulation at the bioelectronics-tissue interface for in-situ stimulation (e.g., neuromodulation) and sensing (e.g., biomarker molecules), necessitating the use of suitable interfacial materials. The electrical process relates to electron transport for signal routing and setting intended operating points (e.g., voltage), requiring the use of highly conductive materials (H. Yuk, B. Lu, X. Zhao, Hydrogel bioelectronics. Chem. Soc. Rev. 48, 1642-1667 (2019). doi: 10.1039/c8cs00595h). From the mechanics standpoint, materials that mimic tissue properties such as stretchability (~20%-75%) and compliance (Young's modulus E below or in the range of kPa to MPa) are needed to establish conformable contact with tissue for high fidelity sensing/stimulation. This requirement is frequently considered for minimizing the tissue function disruption as well as the risk of implant complications resulting from scar formation, lead fracture, and tissue injury (Id.). However, the electrochemical and electrical performance of these materials may be affected by the strain originating from the movement and complex topography of the tissue.

Clinically established interfacial materials with high electrochemical performance for bioelectronics-tissue interfaces (e.g., noble metal-based ones) are all rigid (E>1 GPa) and brittle (fracture strain <1%). Recent advances in soft and stretchable conductors have enabled the development of strain-resilient devices with tissue-like mechanical properties, which maintain their electrical connection despite being extensively stretched (~800%, see Y. Wang et al., Standing Enokitake-like Nanowire Films for Highly Stretchable Elastronics. ACS Nano. 12, 9742-9749 (2018). doi: 10.1021/acsnano.8b05019 and S. Choi et al., Highly conductive, stretchable and biocompatible Ag—Au core-sheath nanowire composite for wearable and implantable bioelectronics. Nat. Nanotechnol. 13, (2018), pp. 1048-1056. doi: 10.1038/s41565-018-0226-8). While these strain-resilient devices are suitable for construction and integration of soft, stretchable, solid-state electronics, when they are applied to electrochemically probe the tissue environment (involving ionic biofluid surrounding), their performance is distorted by strain- or corrosion-related issues. The former issue is manifested in devices with relatively low intrinsic conductivity (e.g., graphene, PEDOT:PSS: 1×10³ S/m), wherein strain-induced changes to their resistance cause significant deviation from the intended operating points (e.g., activation voltage), subsequently corrupting the reactions at the interface (Q. Zhai et al., Vertical Gold Nanowires Stretchable Electrochemical Electrodes. Anal. Chem. 90, 13498-13505 (2018). doi: 10.1021/acs.analchem.8b03423; Y. Liu et al., Soft and elastic hydrogel-based microelectronics for localized low-voltage neuromodulation. Nat. Biomed. Eng. 3, 58-68 (2019). doi: 10.1038/s41551-018-0335-6; J. Zhang et al., Stretchable Transparent Electrode Arrays for Simultaneous Electrical and Optical Interrogation of Neural Circuits in Vivo. Nano Lett. 18, 2903-2911 (2018). doi: 10.1021/acs.nanolett.8b00087). The latter issue is especially observed in devices with highly conductive materials that are rapidly oxidized upon exposure to the surrounding solution (e.g., silver-based nanomaterials), despite applying surface modification (prone to surface defects/pores, see H. Lee et al., Highly Stretchable and Transparent Supercapacitor by Ag—Au Core Shell Nanowire Network with High Electrochemical Stability. ACS Appl. Mater. Interfaces 8, 15449-15458 (2016). doi: 10.1021/acsami.6b04364).

As such, this group of devices cannot sustain the wide voltage range required to perform electrochemical reactions (F. Decataldo et al., Stretchable Low Impedance Electrodes for Bioelectronic Recording from Small Peripheral Nerves. Sci. Rep. 9, 10598 (2019). doi: 10.1063/5.0021887).

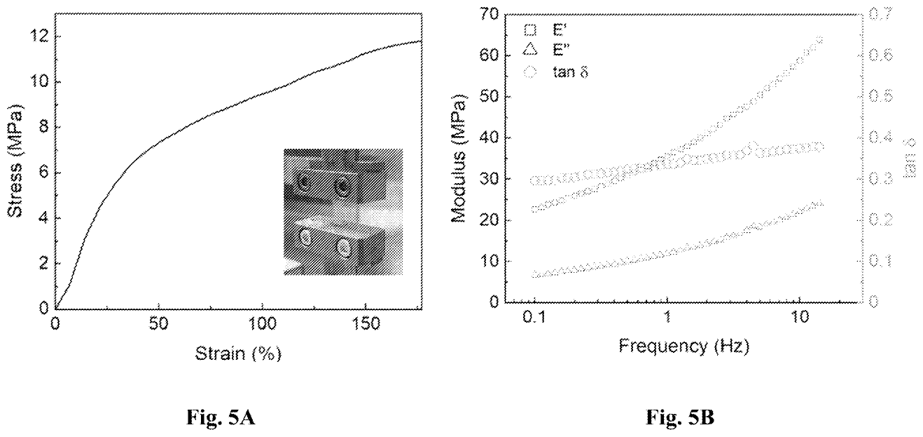

According to certain aspects, the present embodiments provide a layered-architectural composite design that centers on decoupling the bioelectronic materials configuration into an interfacial element for electron transfer and an interconnection element for electron transport. This design allows for exploiting and coupling surface channel cracks (within the brittle interfacial element) and anisotropic out-of-plane/in-plane electron conduction (within the interconnection element) to eliminate strain effects on the device performance. It further allows for a broad selection for the interfacial materials (including brittle noble metal-based materials). Following this approach, created was a functionally diverse library of thin (~140 μm), soft (~10 MPa, FIGS. 5A and 5B), stretchable (>150%), highly conductive (~1 Ω/sq) and strain-insensitive bioelectrodes (SIBs).

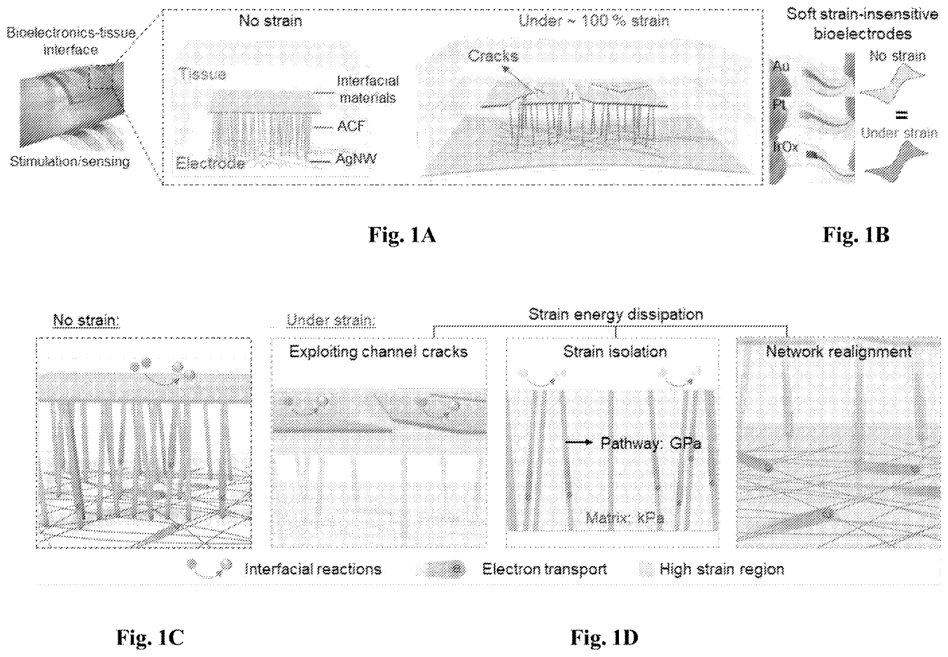

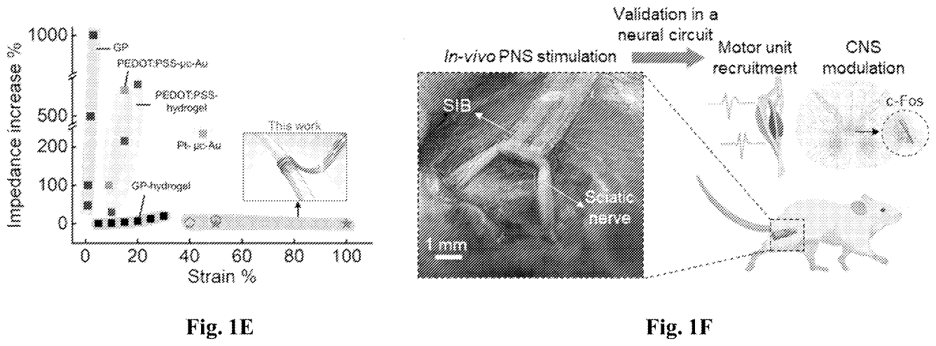

FIGS. 1A to 1F illustrate example aspects of soft strain-insensitive bioelectrode (SIB)—such as architecture, strain dissipation mechanisms and applications—according to embodiments: FIG. 1A is an example illustration of the SIB as a bioelectronics-tissue interface under strain. FIG. 1B illustrates example aspects of brittle noble metal-enabled SIBs for high fidelity stimulation/sensing under strain. FIG. 1C is a schematic presentation of SIB's underlying electron transfer and out-of-plane/in-plane electron transport processes. FIG. 1D illustrates example strain energy dissipation mechanisms within each layer of the SIB. FIG. 1E is an example Ashby diagram of electrochemical impedance (at 1 kHz) increase versus tensile strain. Au-SIB/IrOx-SIB are indicated by star/pentagon markers. FIG. 1F illustrates a representative SIB interfacing the sciatic nerve of a mouse for nerve stimulation to achieve synchronized motor units recruitment and CNS modulation (manifested as c-Fos expression).

One example fabrication strategy according to embodiments such as that shown in FIG. 1A is based on: 1) constructing the tissue-interfacing element as a thin film with favorable electron transfer characteristics; 2) constructing the interconnection element by utilizing intrinsically stretchable and highly conductive AgNW-based traces, which are inlaid in the surface layer of a soft rubbery matrix (poly[urethane acrylate], PUA); and 3) seamlessly integrating the two elements via a thin adhesive and anisotropically conductive film (ACF), which features isolated conductive particles embedded in a stretchable matrix to facilitate anisotropic out-of-plane electrical conduction. A variety of thin film materials, including those that are brittle but have high electrochemical performance (e.g. gold, Au; platinum, Pt; and iridium oxide, IrOx), can be built atop the ACF/AgNW/PUA layers via standard thin-film deposition techniques (FIG. 1B).

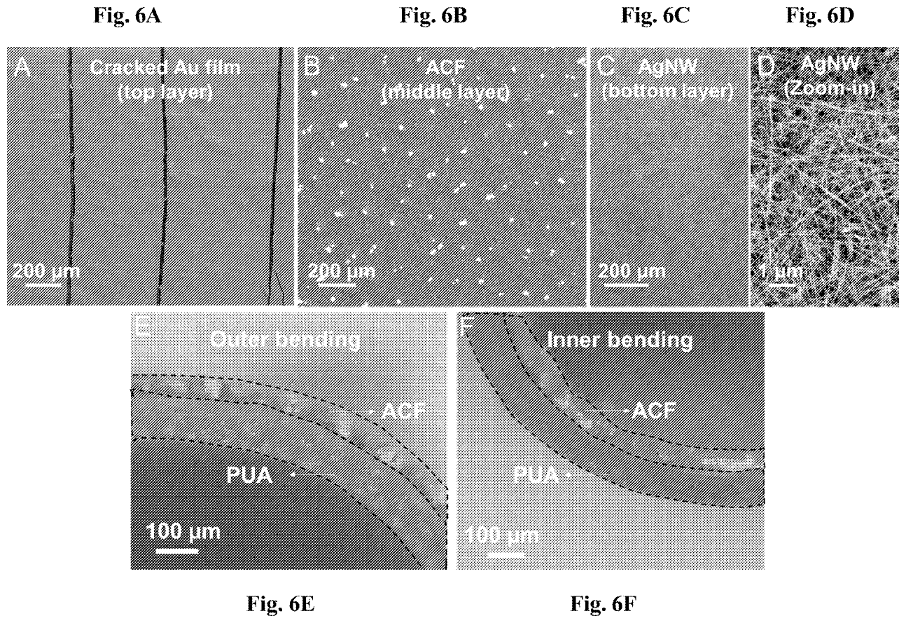

Fundamentally, strain insensitivity of an SIB according to embodiments is governed by three layer-specific strain energy dissipation mechanisms as illustrated in FIG. 1C, FIG. 1D and FIG. 6. These mechanisms include 1) crack channeling of brittle interfacial thin film; 2) strain isolation of the out-of-plane conductive pathway in the ACF layer; and 3) realignment of in-plane AgNW networks. Interfacial channel cracks are exploited as means of tensile strain energy release. Each cracked fragment experiences minimal strain, while remaining electrically connected to the system, preserving the overall active surface area of the SIB. As the electrical bridge between the cracked interfacial thin film and the AgNW/PUA layer, the ACF renders strain-insensitive out-of-plane electrical conduction, since the rigid conductive microparticles (~GPa) are strain-isolated by the surrounding soft matrix (~kPa). The AgNWs in the bottom layer form in-plane conductive percolation networks that are strongly anchored onto the soft PUA substrate via in-situ crosslinking. This strong binding enables network realignment to release strain energy (J. Liang, L. Li, X. Niu, Z. Yu, Q. Pei, Elastomeric polymer light-emitting devices and displays. Nat. Photonics. 7, 817-824 (2013). doi: 10.1038/nphoton.2013.242).

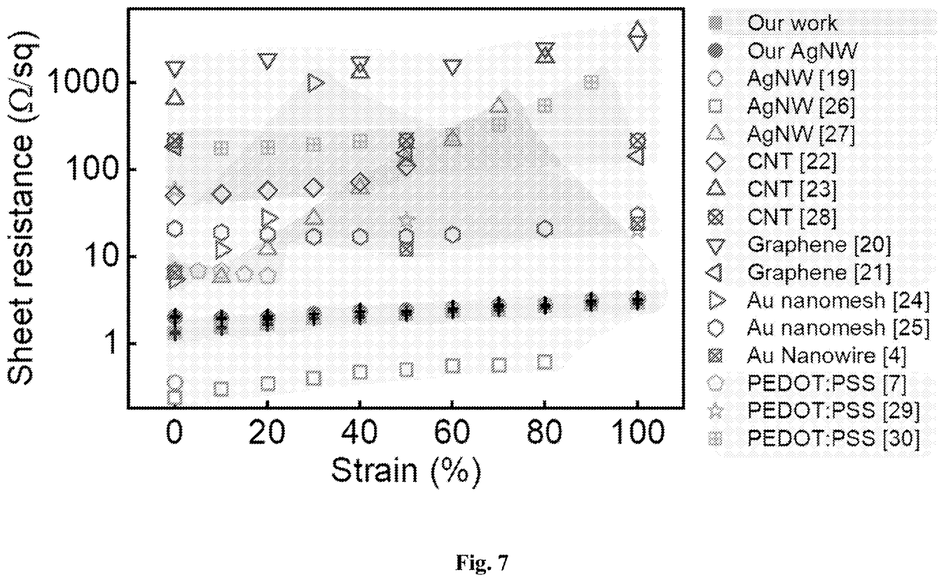

The strain-sensitivity of the electrical/electrochemical performance of the SIBs is compared with those obtained by the state-of-the-art stretchable bioelectrodes (FIG. 1E, FIG. 7, Table S1) (J. Deng et al., Electrical bioadhesive interface for bioelectronics. Nat. Mater. 20, 229-236 (2021) doi: 10.1038/s41563-020-00814-2; I. R. Minev et al., Biomaterials. Electronic dura mater for long-term multimodal neural interfaces. Science. 347, 159-163 (2015). doi: 10.1126/science.1260318). The present SIBs were applied to perform high fidelity sensing of multiple biomarkers and neurostimulation under strain. The in-vivo operation of these SIBs were validated in the context of a neural circuit. Specifically, the bioelectrodes—featuring brittle IrOx layer—were interfaced with the sciatic nerve and the modulation of the bidirectional-interconnecting central nervous system (CNS) and motor unit (within the neural circuit) was verified via peripheral nerve stimulation (PNS).

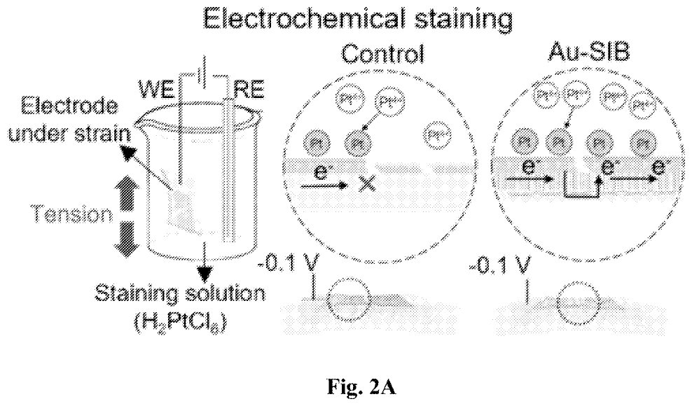

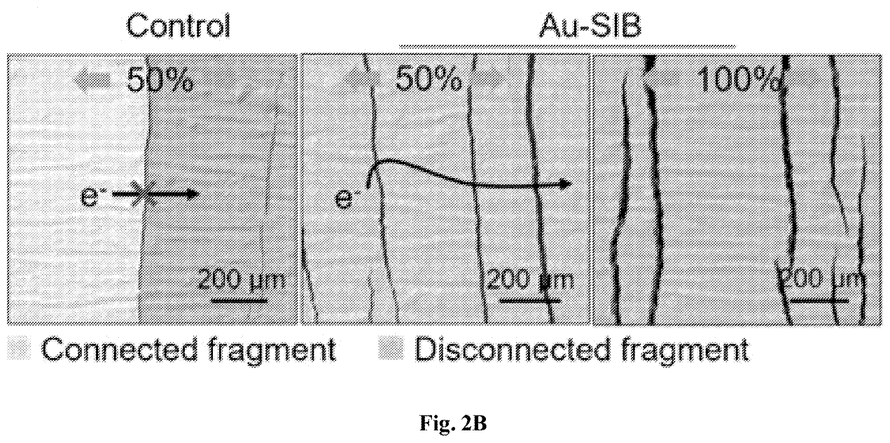

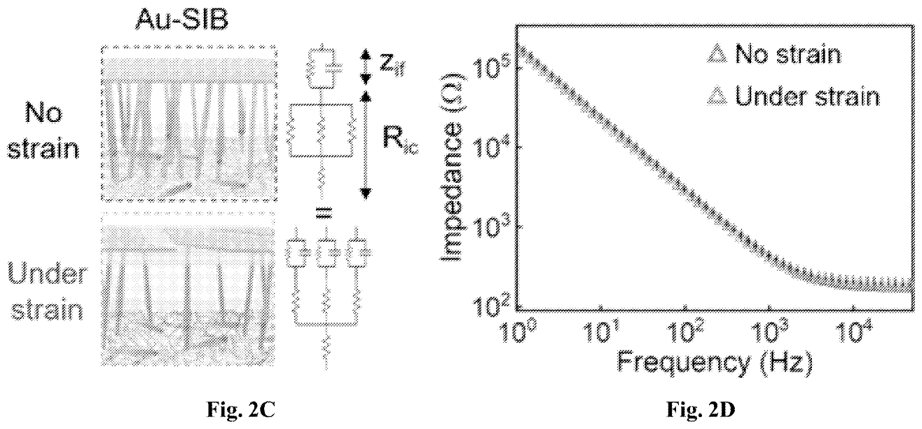

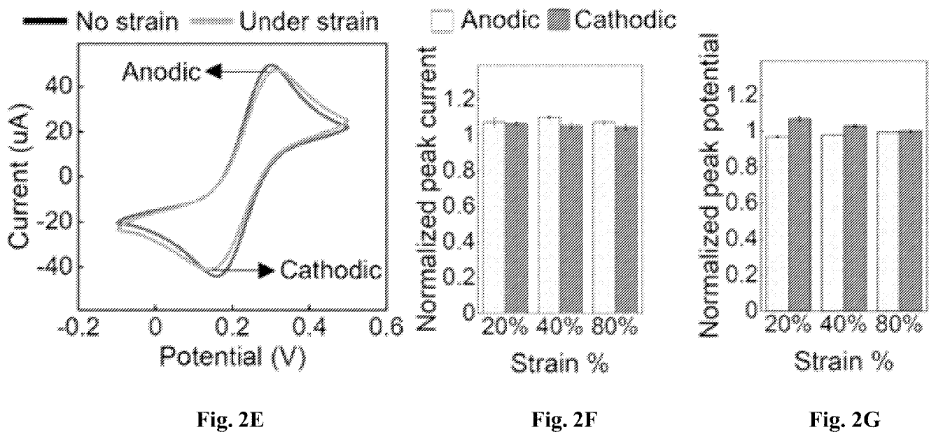

FIGS. 2A to 2G provide example visualization and electrochemical characterization of example Au-SIBs performance under strain according to embodiments. FIG. 2A is an example schematic of the introduced electrochemical staining method. FIG. 2B provides colored SEM images of the electrochemically-stained control (Au/ACF/PUA) electrode and Au-SIB under strain. They illustrate the electrical disconnection of a cracked fragment in the control electrode, while the Au-SIB's cracked fragments remained electrically connected. FIG. 2C provides example corresponding Randles model schematics of unstrained and strained/cracked SIBs. Interconnect resistance is denoted by Ric. Interfacial impedance is denoted by Zif. The SIB design enables the formation of parallel pathways for electron transfer/transport and maintenance of the active surface area under strain. FIG. 2D provides an example EIS comparison of Au-SIBs: unstrained vs. 100% tensile-strained (N=3 measurements by the same device for each strain condition). p=0.12 comparing strained and unstrained measurement groups. Error bars indicate SEM. FIG. 2E provides an example CV comparison of Au-SIBs: unstrained vs. 100% tensile-strained (in K3Fe(CN)₆ solution). FIGS. 2F and 2G illustrate example anodic/cathodic peak positions (F) and heights (G) of Au-SIBs under 20% through 80% strain (normalized to unstrained values; N=3; p values range: 0.053-0.745; error bars indicate SEM).

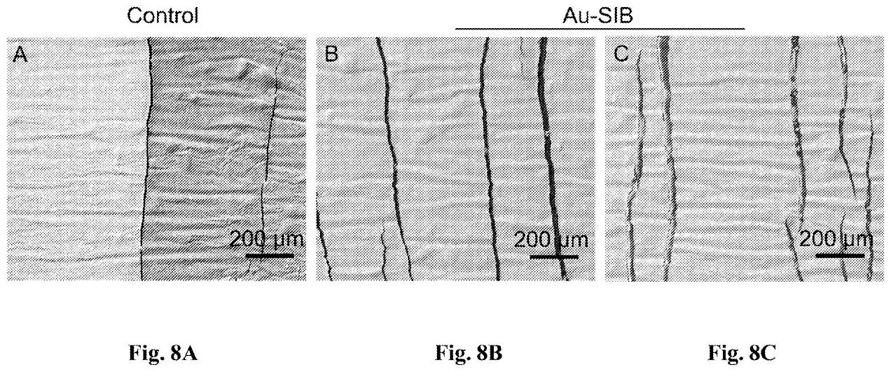

To visualize and quantitatively characterize the SIBs' strain-insensitivity, correspondingly utilized was an electrochemical deposition-based staining method, as well as electrical impedance spectroscopy (EIS) and cyclic voltammetry (CV). Characterized were Au-deposited SIBs (Au-SIBs) as model test devices over a range of tensile strain conditions. The devised electrochemical staining method enables the spatial mapping of the electrical connection of the interfacial thin film. It uses a standard electrodeposition setup to deposit a conductive material (e.g. Pt) onto a test electrode (under strain), wherein only the electrode surface regions that remain electrically connected to the power source support electrodeposition as shown in FIG. 2A. As a result, the electrochemically deposited regions (“stained”) of the test electrodes exhibit a significant imaging contrast under scanning electron microscopy (SEM) as compared to the non-deposited regions (see FIGS. 8A to 8C). FIG. 2B illustrates that the cracked fragments of a representative Au-SIB were fully “stained”, indicating the preservation of their conductivity under varying large strain levels (in contrast to the case of a control Au/elastomer electrode).

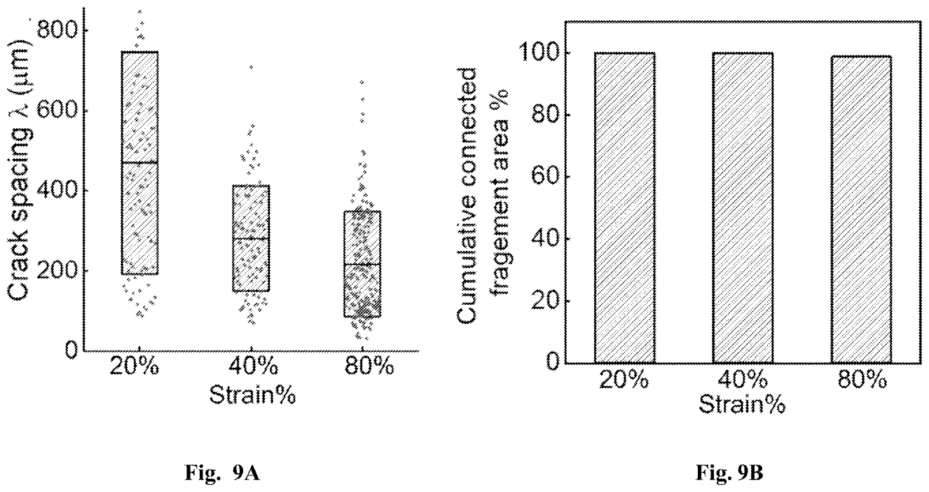

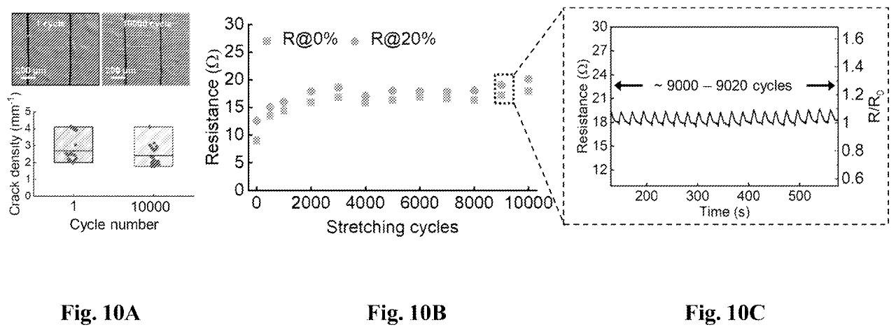

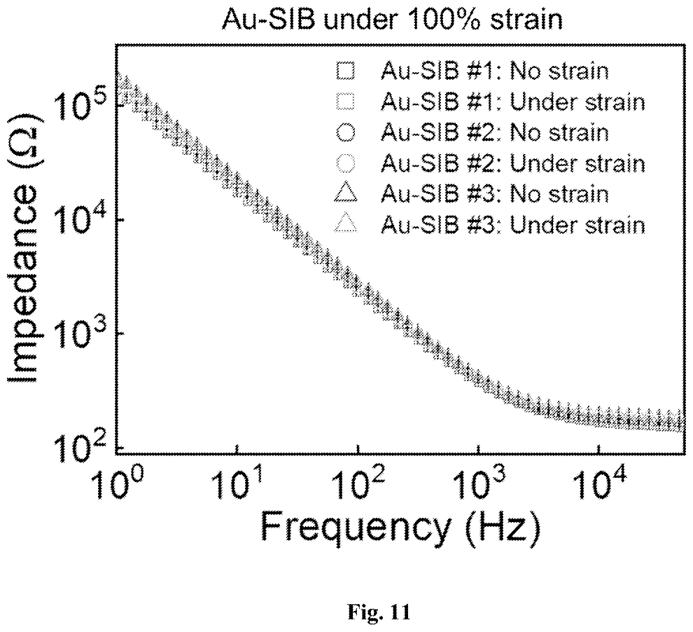

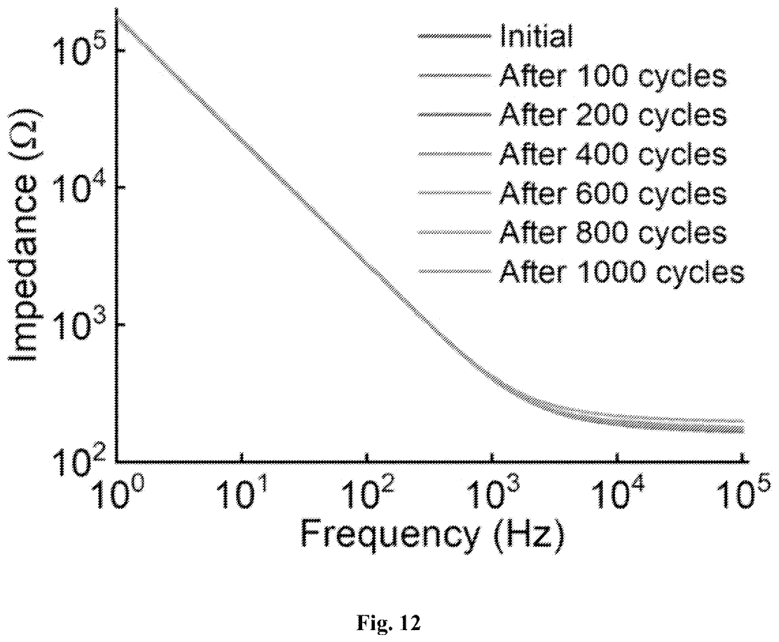

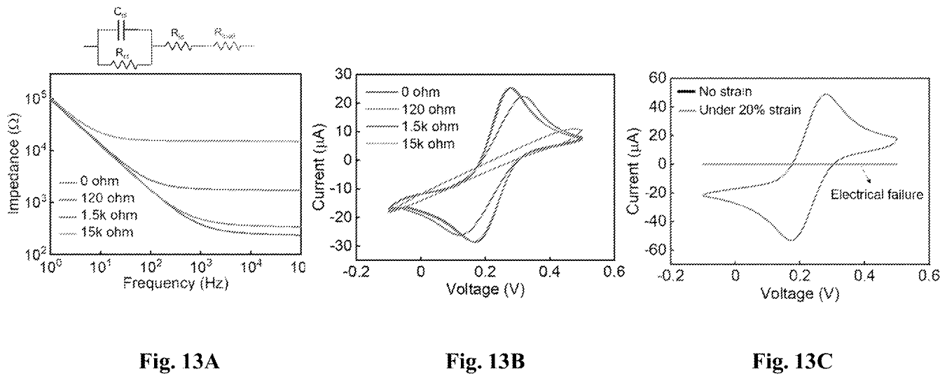

Characterized were the cracked fragments via SEM imaging, and analyzed were the results with the aid of a dedicated statistical model. The results indicated >99% of the brittle interfacial layer's surface area remains connected (FIG. 9B). The cyclic stretching studies also demonstrated that the Au-SIB's crack density does not increase, despite repeated stretching (>10,000 cycles, at 20% strain, FIG. 10A), and that the device's low resistance is minimally impacted during dynamic stretching (FIGS. 10B and 10C, ~2Ω variation). EIS and CV characterization results indicate that the changes to the SIB system's underlying electrochemical and electrical components (modeled by FIG. 2C) are negligible under strain. The impedance spectrum of representative 100%-strained SIBs exhibited statistically identical curves in impedance magnitude (1 Hz-100 kHz), as compared to their unstrained states (FIGS. 2D and 11). Besides, the SIB's electrochemical impedance has minimal change after 1000 cycles of tension (FIG. 12). Furthermore, the CV plots of the unstrained and strained SIBs exhibit near-identical features across 20%-100% strain (FIGS. 2E-2G). Without strain accommodation, tissue-induced strain causes a substantial increase in the interconnect resistance to the point of open circuit, corrupting the electrode's electrical and electrochemical characteristics (manifested as shifting and distortion of the EIS and CV curves, respectively, FIGS. 13A to 13C).

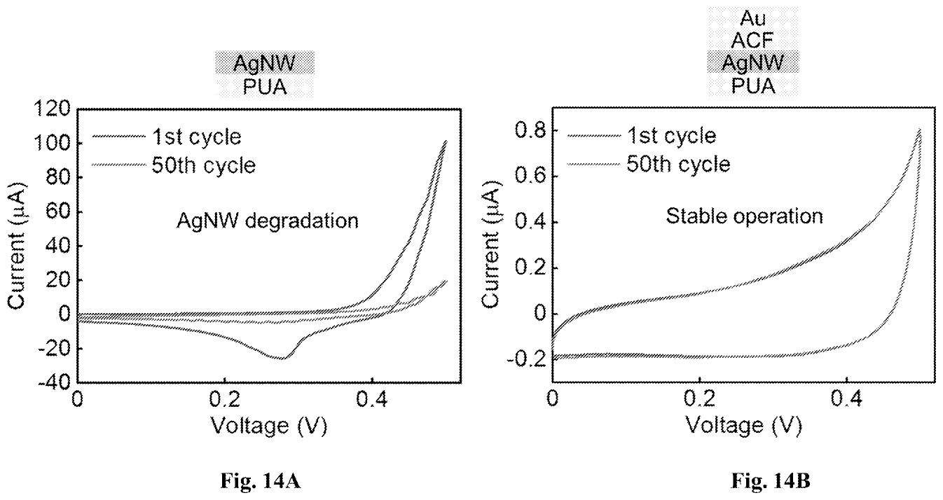

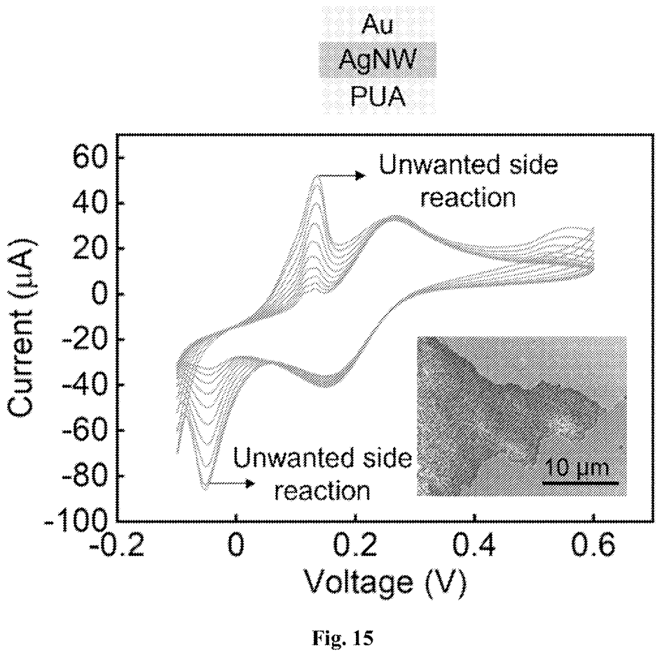

Compared with bare AgNW/PUA, Au-SIB is more electrochemically stable (see FIGS. 14A and 14B). The underlying AgNW/PUA layer of the SIB does not experience galvanic corrosion in the presence of cracking. This contrasts with the case of Au deposited-AgNW/PUA electrodes, which galvanically corrode due to the crack-induced exposure of the AgNWs (FIG. 15). The SIB's corrosion resistance indicates the protective role of the hydrophobic ACF in preventing the AgNWs oxidation and cytotoxic Ag ions release (useful for prolonged and biocompatible in-vivo operations).

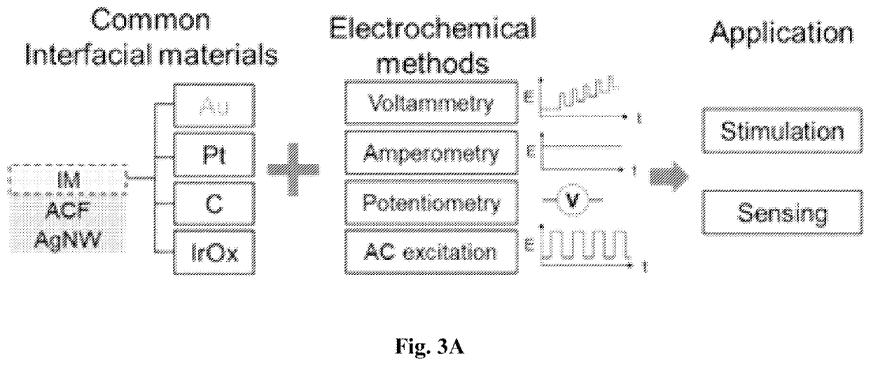

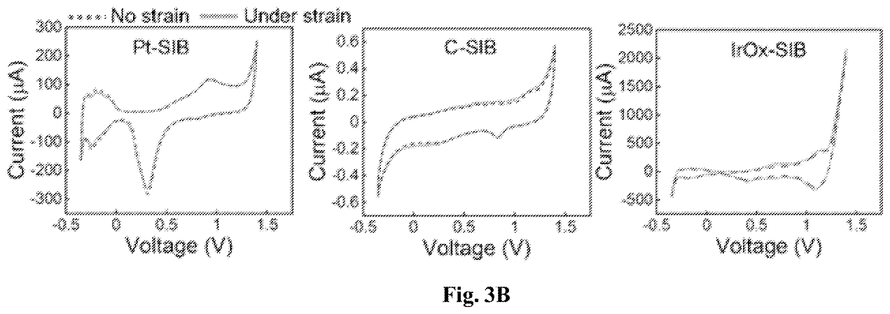

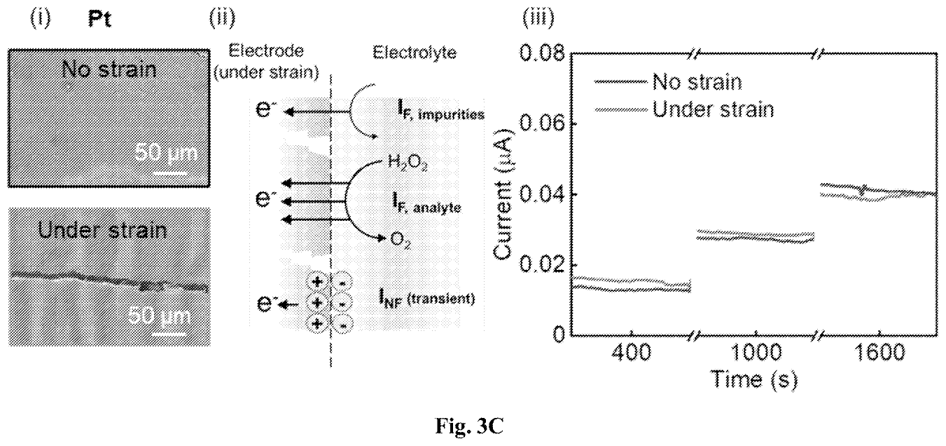

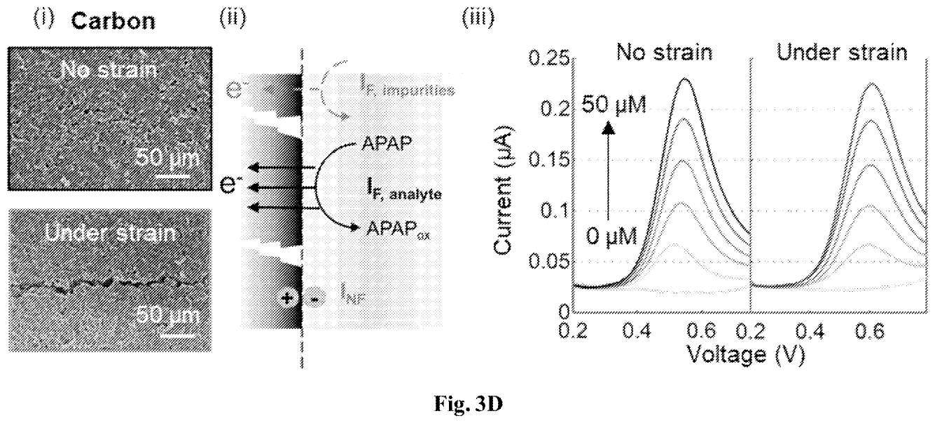

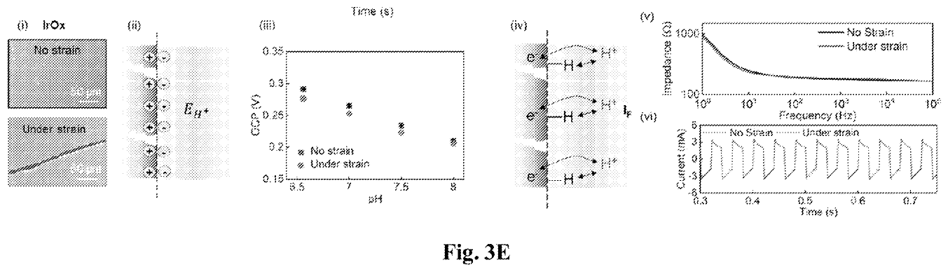

FIGS. 3A to 3E illustrate aspects of an example library of SIBs featuring brittle interfacial materials for electrochemical sensing and stimulation according to embodiments. FIG. 3A is a diagram illustrating example pairing of SIBs with different electrochemical probing methods for diverse applications. IM represents the interfacial material layer. FIG. 3B are graphs providing example CV characteristics of Pt-SIB, Carbon-SIB, and IrOx-SIB at 40% tensile strain vs. unstrained state. FIG. 3C are diagrams illustrating an example Pt-SIB characterization: (i) SEM images of the unstrained and 40%-strained Pt-SIBs; (ii) schematic of the amperometric H₂O₂ sensing process; and (iii) Pt-SIB's H₂O₂ responses (0, 50, 100 μM) at 40% tensile strain vs. unstrained. FIG. 3D are diagrams illustrating an example Carbon-SIB characterization: (i) SEM images of the unstrained and 100%-strained carbon-SIBs; (ii) schematic of the DPV acetaminophen (APAP) sensing process; and (iii) carbon-SIB's APAP responses at 100% tensile strain vs. unstrained. FIG. 3E are diagrams illustrating an example IrOx-SIB characterization: (i) SEM images of the unstrained and 40%-strained IrOx-SIBs; (ii) schematic of the pH sensing process; (iii) IrOx-SIB's pH open circuit potential (OCP) responses at 40% tensile strain vs. unstrained; (iv) schematic of the faradaic process for neurostimulation; and (v, vi) corresponding EIS and ex-vivo neurostimulation characterization of the IrOx-SIBs at 40% tensile strain vs. unstrained.

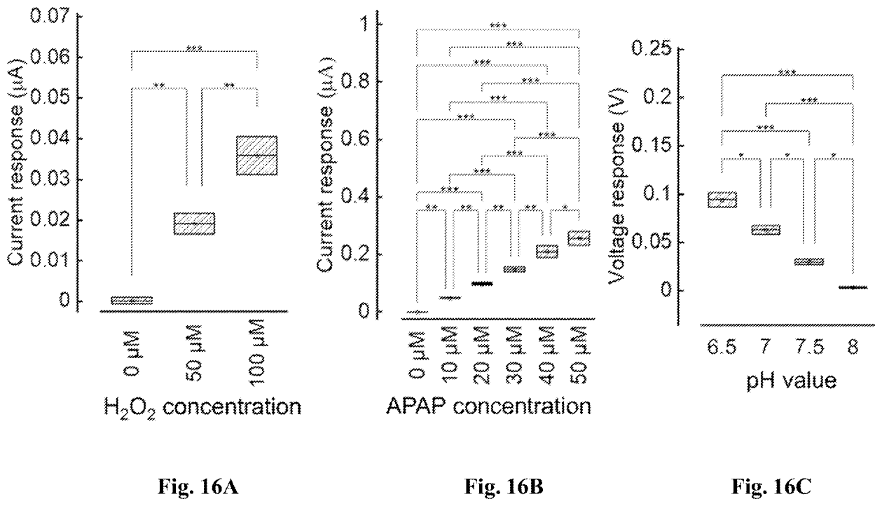

A diverse library of interfacial materials (beyond Au) can be simply incorporated in the SIB structure via standard thin-film deposition techniques and paired with different application-specific electrochemical probing methods involving wide-ranging voltage and frequency conditions as shown in FIG. 3A. Deposited were Pt, carbon, and IrOx onto ACF, as examples of commonly-used brittle interfacial materials, leading to a series of SIBs with strain-insensitive interfacial electrochemical reaction features (validated via CV characterization, as shown in FIG. 3B). These electrodes (exhibiting minimal device-to-device variation, see FIGS. 16A to 16C) were applied under large strain to carry out amperometry, voltammetry and potentiometry, representing distinct electron-transfer processes as shown in FIGS. 3C to 3E.

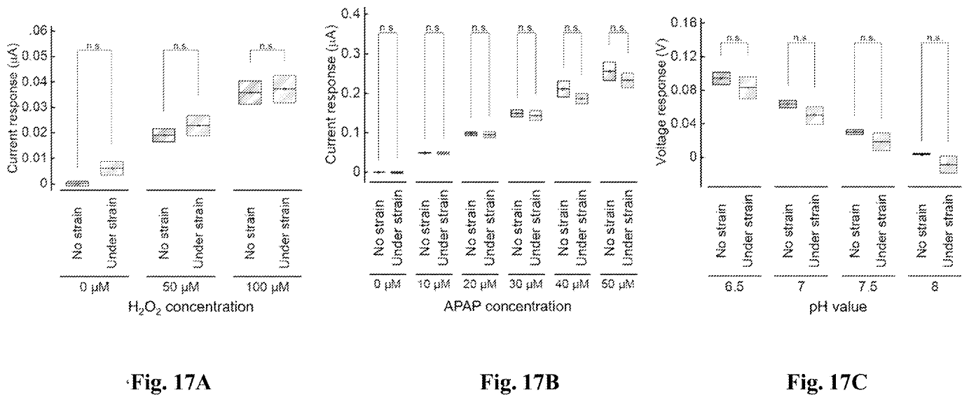

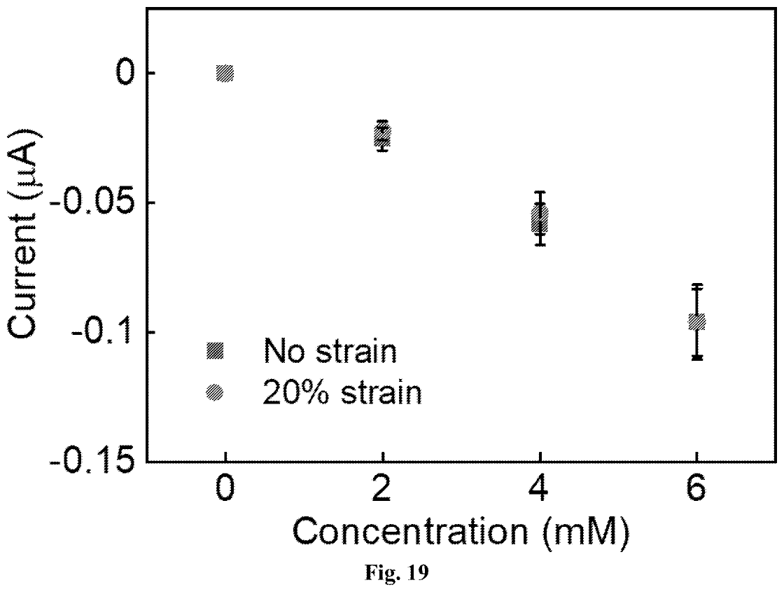

The choice of Pt as an interfacial material was motivated by its common use as an H₂O₂ sensing layer in oxidase-based enzymatic biosensors. As shown in FIGS. 3C, 17A, and 18A, the corresponding amperometric responses of the Pt-SIB at its relaxed vs. tensile states were similar for different concentrations of H₂O₂. These results indicate that the faradaic current was minimally affected by the strain despite the occurrence of the cracks. Strain-insensitive H₂O₂-sensing is also observed when using Au as an interfacial material (see FIG. 19). The choice of carbon was motivated by its common use in the quantification of low concentration electroactive biomarkers. Here, investigated was the effect of strain on the response of the carbon-SIB electrodes in the context of differential pulse voltammetry (DPV) for the detection of acetaminophen. As shown in FIG. 3D (iii) and FIG. 17B, the corresponding DPV peak current measured by the electrode at its relaxed vs. tensile states were similar for different concentrations of acetaminophen. The strain-induced shift in redox potential was minimal (~0.1 V).

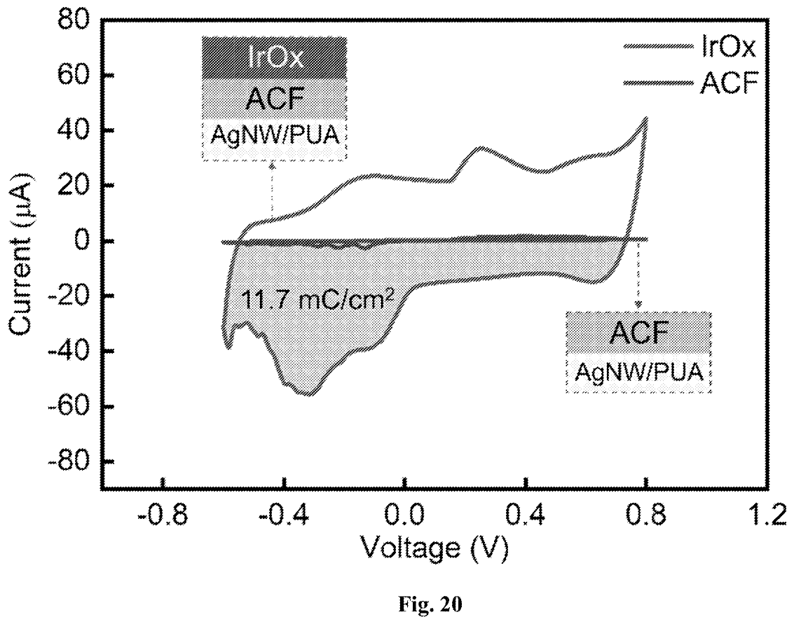

The choice of IrOx as an interfacial material was motivated by its significant yet unrealized full potential in bioelectronics. IrOx is not only biocompatible, but also facilitates fast reversible redox reactions with surrounding hydrogen ions (C. Boehler, S. Carli, L. Fadiga, T. Stieglitz, M. Asplund, Tutorial: guidelines for standardized performance tests for electrodes intended for neural interfaces and bioelectronics. Nat. Protoc. 15, 3557-3578 (2020). doi: 10.1038/s41596-020-0389-2), enabling large and reversible current injection. The latter point particularly makes IrOx superior over noble metals such as Au and Pt for tissue bioelectronics applications such as pH sensing and neural stimulation. However, because of its intrinsic brittleness, IrOx cannot be easily engineered as an intrinsically stretchable electrode. A fabrication strategy according to embodiments enabled simple engineering of IrOx-SIBs where the tight bonding of the IrOx film (supporting electron transfer) to the underlying layer (supporting electron transport) ensured robust device operation despite IrOx's brittleness. The fabricated IrOx-SIB possesses charge storage capacity of ~12 mC/cm² (see FIG. 20), which is similar to the charge storage capacity of the standard clinical stimulation electrodes (S. J. Wilks, S. M. Richardson-Burns, J. L. Hendricks, D. C. Martin, K. J. Otto, Poly (3,4-ethylenedioxythiophene) as a Micro-Neural Interface Material for Electrostimulation. Front. Neuroeng. 2, 7 (2009). doi: 10.3389/neuro.16.007.2009).

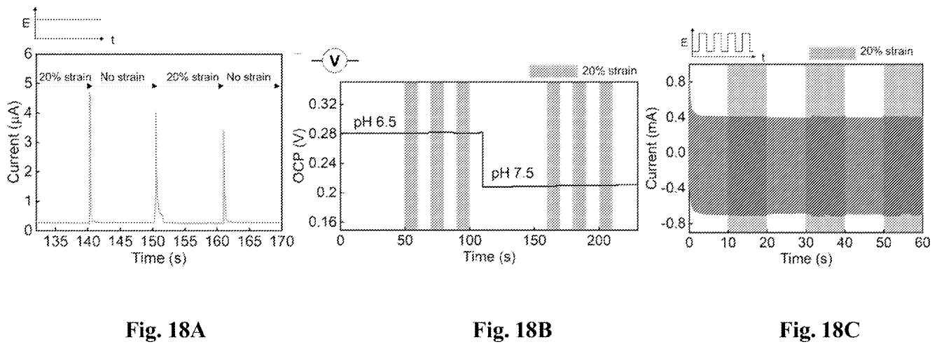

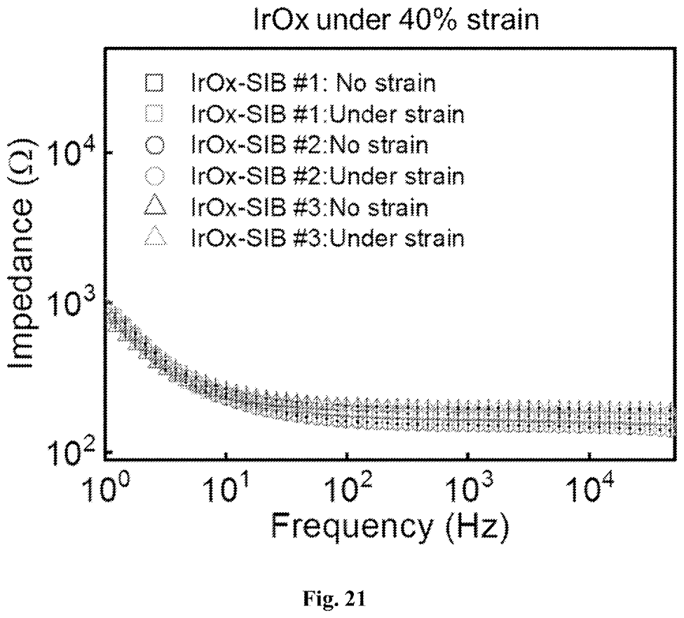

Investigated was the IrOx-SIB for potentiometric pH sensing (FIG. 3E (ii)) and AC excitation (in the form of biphasic pulses, for neurostimulation, FIG. 3E (iv)). The open circuit potential measurements indicate that our IrOx-SIB responses are correlated with the tested solution's pH levels, and that the electrode responses remain unperturbed under both static and dynamic tensile states (FIG. 3E (iii), FIG. 17C and FIG. 18B). As plotted in FIG. 3E (v) and FIG. 21, the EIS measurements performed with an unstrained SIB vs. 40%-strained IrOx-SIBs were similar, illustrating its strain-insensitivity over a wide frequency range, and informing its utility for both low- and high-frequency stimulation settings such as neuromodulation. Ex-vivo neurostimulation characterization studies demonstrate the SIB's robustness in delivering stable current under both static and dynamic tensile conditions (FIG. 3E(vi) and FIG. 18C).

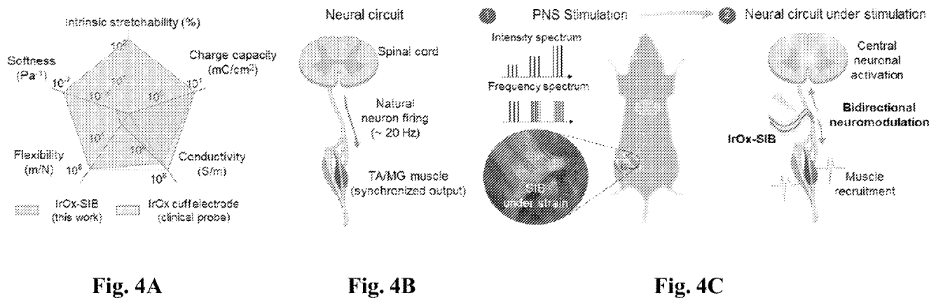

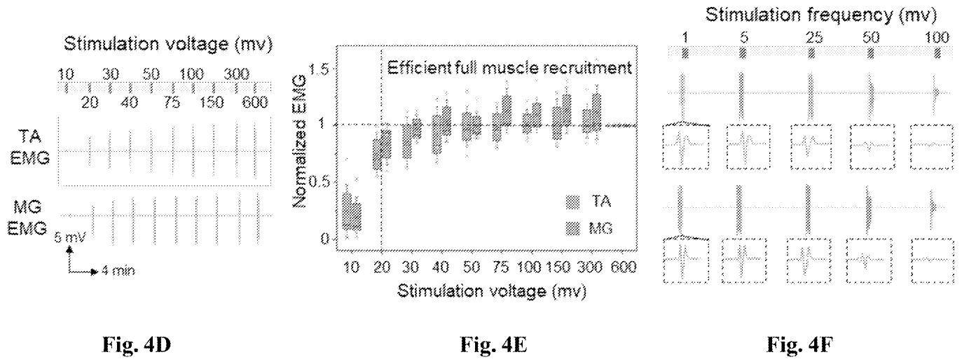

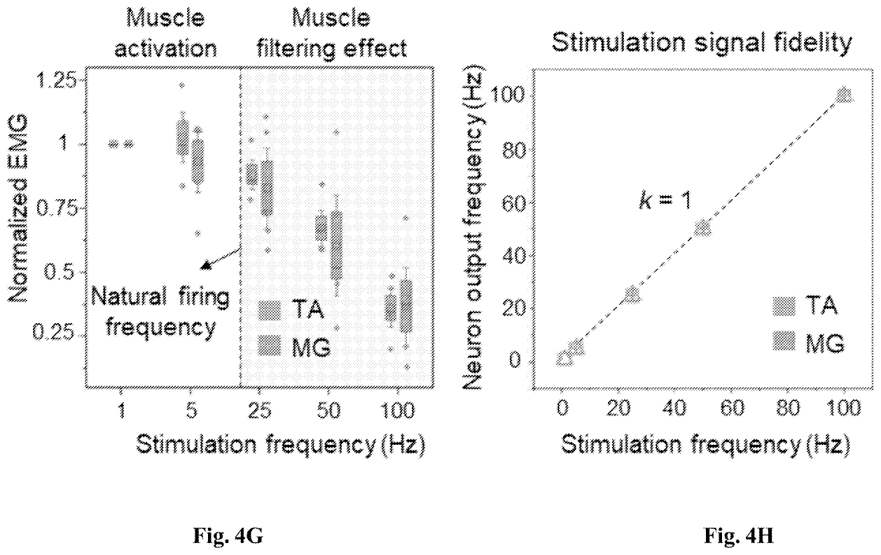

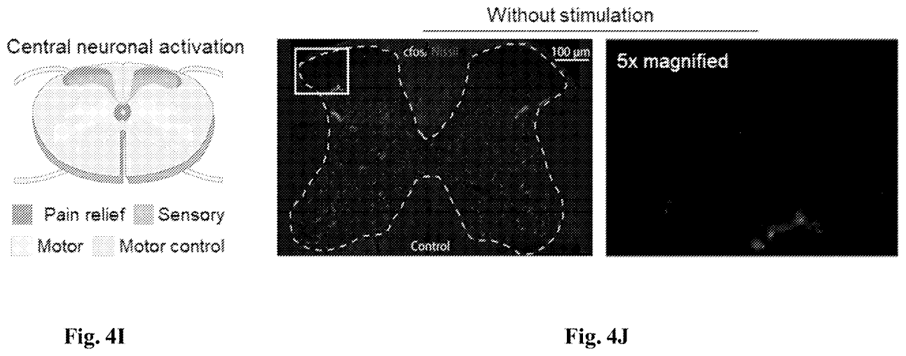

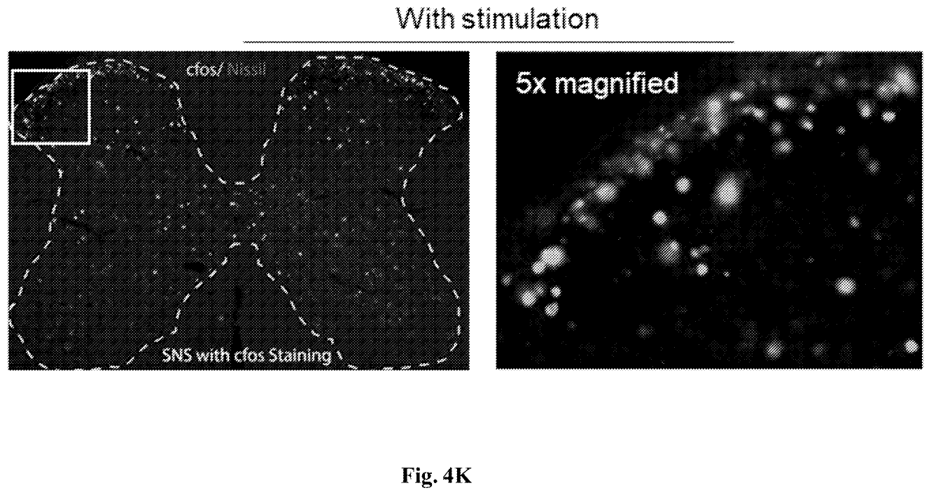

FIGS. 4(A) to 4(K) illustrate example aspects of In-vivo neuromodulation via stimulation of the sciatic nerve by IrOx-SIB under deformation according to embodiments: FIG. 4A is an example Radar chart of electrochemical, electrical and mechanical properties of IrOx-SIB vs. a standard IrOx cuff electrode. FIG. 4B is an example illustration of the neural circuit of a mouse. FIG. 4C illustrates example In-vivo evaluation of IrOx-SIB neuromodulation performance in an established neural circuit model. Peripheral nerve stimulation responses were monitored through TA/MG recordings and central nervous system neuromodulation was evaluated via c-Fos staining of the lumbar spinal cord. FIGS. 4D-4G illustrate example sciatic nerve stimulation with different stimulation voltages at 1 Hz (D-E) and frequencies at 100 mV (F-G), and the correspondingly induced TA and MG muscle EMG recordings (D and F) and their respective normalized amplitudes (E and G). FIG. 4H is an example comparison of the frequency of TA and MG neural signal outputs captured in G versus the stimulation frequency. FIG. 4I provides example schematics of regions determined by their neural functions within the spinal cord. FIGS. 4J and 4K provides example c-Fos expression patterns within lumbar spinal cord with (K) and without (J) sciatic nerve stimulation. Whole spinal cord staining (c-Fos single channel) images and zoom-in views of the selected regions are shown. Dark blue color: nissil staining. Cyan color: c-Fos staining.

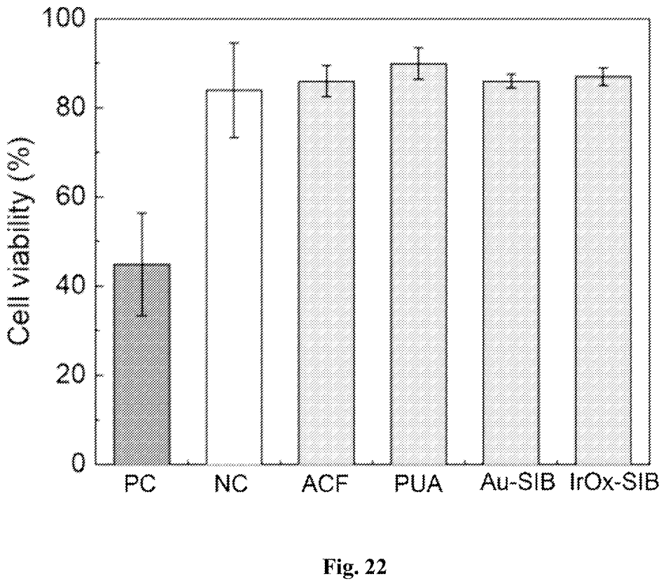

Summarized in FIG. 4A, the IrOX-SIB presents near-identical electrochemical and electrical properties of that of the clinically-used rigid cuff electrodes, while exhibiting tissue-like mechanical properties. The neural tissue-like mechanical characteristic of IrOx-SIB is advantageous over existing implantable electrodes that utilizes rigid materials for neuromodulation: it potentially minimizes scar formation and immune response while rendering efficient stimulation even under deformation (1). To evaluate the performance of IrOx-SIBs during in-vivo stimulation, these bioelectrodes were interfaced with the sciatic nerve to modulate the bidirectional-interconnecting spinal cord and muscles within the neural circuit (see FIG. 4B). Accordingly, after validating its biocompatibility via cellular viability studies (see FIG. 22), the IrOx-SIB was applied in living mouse studies to deliver clinically-relevant voltage and frequency stimulation conditions.

Concurrently recorded were the real-time electromyography (EMG) of the bilateral anterior tibialis (TA) and medial gastral (MG) muscles innervated by the sciatic nerve as shown in FIG. 4C. To validate CNS neuromodulation, the subsequently expressed c-Fos protein levels (induced by sciatic nerve stimulation) in the lumbar spinal cord were labelled via immunofluorescent staining (FIG. 4C). The sciatic nerve stimulation with increasing voltage levels (at 1 Hz) led to the increase, followed by the saturation, of the TA and MG EMG intensities as shown in FIGS. 4D and 4E). This trend indicates the increase in the synchronized recruitment of the available motor units, up to the point of full recruitment, which is achieved at potential levels as low as ~20 mV (illustrating efficient recruitment). As shown in FIGS. 4F and 4G, the present studies involving sciatic nerve stimulation with excitation frequencies spanning from 1-100 Hz (at 100 mV) revealed the low pass filtering characteristic (with cut-off frequency at ~20 Hz) of the muscle contraction (H. J. Freund, Motor unit and muscle activity in voluntary motor control. Physiol. Rev. 63, 387-436 (1983). doi: 10.1152/physrev.1983.63.2.387). Despite being strained, the example device of embodiments induced TA/MG EMG with similar levels of signal-to-noise ratio (SNR, ~30 dB) as those produced by conventional electrodes (~10-38 dB at ~1 Hz) in unstrained settings (S. Kim, L. K. Jang, M. Jang, S. Lee, J. G. Hardy, J. Y. Lee, Electrically Conductive Polydopamine-Polypyrrole as High Performance Biomaterials for Cell Stimulation in Vitro and Electrical Signal Recording in Vivo. ACS Appl. Mater. Interfaces. 10, 33032-33042 (2018). doi: 10.1021/acsami.8b11546; P. Sabetian, Improved Peripheral Nerve Recording with a Small Form-factor Nerve Electrode; A Novel Bipolar Nerve Cuff Design (2015)).

FIG. 4H illustrates a 1:1 ratio between the applied stimulation frequencies and the recorded TA and MG muscle contraction frequencies. These results demonstrate the fidelity of the neural stimulation and the robustness of the strained SIB in supporting undistorted electron transfer and transport processes. From the standpoint of CNS neuromodulation (see FIG. 4I), the c-Fos proteins were observably expressed in response to the sciatic nerve stimulation, as shown in FIG. 4K (non-stimulated control: FIG. 4J). Furthermore, staining results indicated the spatial-concentration of the c-Fos expression in the dorsal horn of the spinal cord, which is related to the neural processing of sensory information such as pain.

In summary, harnessed was the superior electrochemical stability of diverse interfacial materials and electrical conductivity of silver-nanowire-without being limited by their inherent limitations in stretchable bioelectronic settings (i.e., interfacial materials' crack onset strain and silver-nanowire's electrochemical instability). These materials were combined via a strain-insensitive and anisotropic ultra-conductive film. Simultaneously achieved were strain-insensitivity, high conductivity, and electrochemical stability with a diverse of interfacial materials. On a broader level, by unlocking the mechanical constraints, the design principle allows for combining other materials in ways to harness their superior properties in different domains. This allows for attaining maximum achievable performance offered across constituent materials.

### Example Materials and Methods

### Preparation of Silver Nanowire (AgNW)/Poly (Urethane Acrylate) (PUA)

A dispersion of AgNWs in isopropanol (concentration: 2 mg/ml) was coated on glass substrates using a Meyer rod (RD Specialist) or airbrush (Paasche). The resulting conductive coating on the glass substrates was then coated with a precursor solution consisting of 100 weight parts urethane acrylate (UA, CN990), 20 parts ethylene butyl acrylate (EBA, SR540) and 1-part dimethylolpropionic acid (DMPA, Sigma-Aldrich). The coatings were cured on a Dymax ultraviolet curing conveyor equipped with a 2.5 W/cm² Fusion 300S type ‘H’ ultraviolet curing bulb, at a speed of 0.9 feet per minute for one pass, and then peeled off as a freestanding unit.

### Construction of Strain-Insensitive Bioelectrodes (SIBs)

An anisotropic conductive film (9703, 3M, 50 μm, ACF) was laser cut into the same size as the fabricated AgNW-PUA unit and then transferred onto the AgNW-PUA unit to form the base electrode. Next, the ACF top surface was covered with a mask, which was made by laser-patterning of the ACF's original liner. A 200 nm-thick gold (Au) layer was deposited onto the ACF/AgNW/PUA electrode via e-beam evaporation. All electrochemical methods were performed by a potentiostat (CHI E660). To construct platinum (Pt)-SIBs for electrochemical staining and sensing experiments, platinum nanoparticles (PtNP) were deposited onto the Au/AgNW/PUA electrodes via chemical reduction in an aqueous solution of 2.5 mM H₂PtCl₆ (Sigma-Aldrich) and 1.5 mM formic acid (Sigma-Aldrich) at −0.1 V (vs. Ag/AgCl) for 10 min. To construct iridium oxide (IrOx)-SIB, IrOx was electrodeposited on Pt/Au/ACF/AgNW/PUA by cyclic voltammetry (CV, 0-0.6 V vs. Ag/AgCl, 50 mV/s, 100 cycles) in the prepared aqueous solution. The solution contains 4.5 mM iridium tetrachloride (Sigma-Aldrich), 1% (v/v) hydrogen peroxide (30% wt., Sigma-Aldrich) and 55.5 mM oxalic acid dihydrate (Sigma-Aldrich) with a pH of 10.5, titrated by potassium carbonate (Sigma-Aldrich). The obtained solution was allowed to stabilize for at least 48 h before use. The properties of IrOx-SIBs are compared with commercialized cuff electrodes (MicroProbes Inc.). For carbon-SIB fabrication, the ACF/AgNW/PUA electrode with a laser-patterned liner was first affixed to a glass substrate for spin coating. A small amount of carbon ink (Ercon, E3449) was transferred onto the top ACF surface and spin coated at 4000 rpm, 60 s. Then the electrode was baked at 60° C. for 10 mins.

### Electrical Characterization of SIBs

Samples were fixed onto a linear motion stage. A multimeter was used to directly probe the silver nanowire contact pad to obtain the resistance value of samples. Sheet resistance was calculated based on the measured length and estimated width of samples (assume Poisson's ratio=0.2) under different strain conditions.

### Electrochemical Characterization of SIBs Under Different Strain Conditions

A linear motion stage was used to apply the desired strain levels to the SIBs. For static tensile strain studies, the SIBs' tensile strain states were preserved with the aid of a customized rigid frame, before transferring them from the stage into a beaker for electrochemical characterization. In this setting, Ag/AgCl and Pt were used as the reference and counter electrodes. Paired t-test was used to investigate statistical significance of sensing/CV results (with only one variable) and the two sample K-S test was used to investigate statistical significance of EIS curves across measurement groups. For dynamic tensile strain studies, electrochemical characterizations were performed with the SIBs affixed to the linear motion (applying dynamic strain levels), while ensuring the preservation of the fluidic connection with an Ag/AgCl electrode. The performed electrochemical characterization methods are detailed below:
EIS characterization: the impedance spectrum of Au- and IrOx-SIBs (w/and w/o strain) were measured by applying a 10 mV-amplitude sine wave at different frequencies (1-105 Hz) in a beaker containing a phosphate-buffered saline (PBS) solution.
CV characterization: for Au-SIBs (w/and w/o strain), current signals were recorded by sweeping the voltage from −0.1 V and 0.5 V in 0.1 M KCl solution containing 5.0 mM K3Fe(CN)6 with scan rate of 50 mV/s. For Pt-, carbon-, and IrOx-SIBs, current signals were recorded by sweeping the voltage from −0.3 V and 1.4 V in 0.1 M H₂SO₄ solution at the scan rate of 100 mV/s.
Amperometry characterization: to characterize the H₂O₂ sensing capability of Pt-SIBs (w/and w/o strain), amperometric measurements were performed at +0.3 V. Responses were continuously recorded by a potentiostat under constant sample stirring. The calibration plots were constructed by spiking the sample (PBS) with different concentrations of H₂O₂.
Differential pulse voltammetry (DPV): to characterize the acetaminophen (APAP) sensing capability of carbon-SIBs (w/ and w/o strain), DPV measurements were performed in a beaker system. Voltage was scanned from +0.2 V to +0.8 V (increments: 5 mV, amplitude: 50 mV, pulse width: 0.05 s, sampling width: 16.7 ms, pulse period: 0.5 s).
Potentiometry (open circuit potential): to characterize the pH sensing capability of IrOx-SIBs (w/and w/o strain), OCP measurements were performed using pre-titrated droplets (prepared via introducing NaOH/HCl into PBS), with pH levels spanning from ~6.5 to 8.
Biphasic potential pulses stimulation: to characterize the ex-vivo nerve stimulation capability of IrOx-SIBs (w/and w/o strain), a series of +0.5 V to −0.5 V biphasic potential pulses (f=25 Hz) were continuously applied to the IrOx-SIBs. The current was continuously recorded, while the IrOx-SIBs were immersed in the PBS solution.
Charge storage capacity (CSC) characterization: the CSC of the IrOx-SIB and ACF/AgNW/PUA electrodes were characterized by performing cyclic voltammetry (0.6 V to +0.8 V, at 50 m V/scan rate) in a 0.1 M KCl solution.

### In-Vitro Biocompatibility Test

The cell toxicity was evaluated based on the mouse embryonic fibroblasts system (MEFs). MEFs were isolated from female embryos using a Pierce™ Mouse Embryonic Fibroblast Isolation Kit (Fisher, #88290), from pregnant female C57BL/6 mice with no genetic modifications. As-fabricated devices or components were sterilized (UV for 15 mins) before being incubated in the medium (Dulbecco's Modified Eagle Medium) for 10 days under 37° C. (CO₂ incubator). The positive control group was designed by the addition of AgNO₃ (Sigma-Aldrich) into the medium to reach a concentration of 10 μg/mL. After the devices or components were removed from the medium, the MEFs were cultured in the conditioned medium added with 10% fetal bovine serum for 24 hours. To quantify the viability, the cells were resuspended in Trypan blue stain, loaded into a hemocytometer, and counted manually under a microscope. Each study involved two biological replicates and two technical replicates.

### In-Vivo Sciatic Nerve Stimulation

Animal: six mixed gender, body weight 20-28 g, 3-4-month-old C57BL/6J (C57BL/6J, Catalog No: 000664, The Jackson laboratory, ME USA 04609) mice were used in this study. All animal studies were performed according to the protocols approved by the University of California, Los Angeles Animal Research Committee, under the ARC protocol number 2019-019. The experimental sample size (5 mice in total) was determined by the similar study. The methods were carried out in accordance with the relevant guidelines and regulations in full compliance with the ARRIVE (Animal Research: Reporting of in vivo Experiments) guidelines 2.0.

### Sciatic Nerve Stimulation:

Before each study, animals were weighed to make sure that their body weights were over 20 grams. Mice were anaesthetized by isoflurane via inhalation. The induction of the anesthesia was done with 3-5% isoflurane no longer than 2 min and the maintenance of the anesthesia was done with 0.5-2% isoflurane. Toe pinch was used to validate the state of anesthesia every 15 min throughout the entire procedure. The animals were placed on its side and the other hindlimb was put on a small cotton ball with adhesive tapes to keep the hindlimb stable. We found the femur using the forefinger and made an incision of approximately 0.5 cm, parallel to the femur and approximately 1.5 mm anterior to the femur. The muscles close to the femur were separated with a pair of blunt-tip forceps without cutting the muscles or nerves. The muscle layers could be separated easily without any bleeding and the sciatic nerve was then visible. In case of bleeding, we used a cotton-tipped swab to absorb the blood. Once the sciatic nerve was exposed, the IrOx-SIB was gently placed underneath across the main branch of the sciatic nerve to deliver the electrical stimulation. 5s-electrical stimulation pulses (pulse width: 10 ms, at least 5 repeats for each stimulation, resting period between stimulations: 1 min) were delivered to the sciatic nerve with either varying intensity or frequency in a randomized sequence. For stimulation data, it describes both technical replicates and biological replicates.

### Electromyography (EMG) Recording:

The EMG recording was used to monitor and evaluate the muscle activities in response to the sciatic nerve stimulation. For this purpose, tibialis anterior (TA) and medial gastrocnemius (MG) muscles of anesthetized mice were exposed for EMG recording. The recording electrode wires were inserted to the muscles using a 27 g needle, while the ground electrodes were placed on the ear of the animals. EMG signals were recorded throughout each stimulation or sham session with three phases of testing 1) pre-stimulation baseline (3 minutes), 2) sham or stimulation (5s), and 3) post-stimulation baseline (6 minutes). After the procedure, the animal was euthanized through 4% paraformaldehyde transcardiac perfusion.

### Tissue Processing and Immunofluorescence Staining:

Spinal cord tissues of the euthanized animal were sectioned coronally on a Leica CM1900 cryostat at 30 μm. All tissue slices were processed by 10% normal donkey serum for an hour at room temperature. The spinal cord was also stained for antibodies against c-Fos (ab 190289, Abcam, RRID: AB_2737414), CaMKII (MAB8699, Millipore, Millipore, RRID: AB_2067919), and pCREB (sc-7978, Santa Cruz, RRID: AB_2086020). Fluorescent conjugated secondary antibodies (488 anti-mouse, Catalog No: 715-547-003), Cy3 anti-rabbit (Catalog No: 711-165-152), Jackson Laboratory, Bar Harbor, ME) were incubated with the tissue at room temperature for two hours in place of the aforementioned biotinylated secondary antibodies. After washing with PBS/T (Triton), the slides were mounted in Vector DAPI (Catalog No: H-1200) mounting solution (Vector Labs, Burlingame, CA) and examined using fluorescence microscopy (Echo Revolve).

Derivation of Probability that a Cracked Fragment in the Interfacial Layer Remains Electrically Connected Via at Least One Conductive Element in the ACF Layer

To set up the problem, denoted was a representative cracked fragment area as a, the total interfacial area as A, and the density of conductive elements within ACF as m. Following a simple binomial distribution and a conservative assumption that the surface area of the conductive elements can be neglected, the probability P that the cracked fragment in the interfacial layer overlaps with at least one underlying conductive element can be expressed as:

$$PP = 1 - {( 1 - \frac{aa}{AA} )}^{\text{?}}$$

$$\text{?} \text{indicates text missing or illegible when filed}$$

In the present context, A is 4 mm² and m is on average 106 particles per mm². Thus, the probably that the cracked fragment remains electrically connected can be calculated as:

$$PP = 1 - {( 1 - \frac{aa}{AA} )}^{424}$$

Utilized was this probability expression to set the threshold for determining the electrical connection status of the fragments (here, 95%). By empirically characterizing the cracked fragments' sizes formed under different strain conditions (20%, 40%, and 80%) via SEM imaging, and applying the set threshold, estimated was the percentage of cumulative connected cracked fragments with respect to the original (uncracked) interfacial area (see FIG. 9B).

TABLE S1
Comparison with the state-of-the-art stretchable conductors and bioelectrodes.

| | | | | | | Vertically- |
|---|---|---|---|---|---|---|
| Parameter | SIB | AgNW | Graphene | CNT | Nanomesh | aligned AuNW |
| Sheet | Low (1.33) | Low (0.36) | High (10²- | High (10¹- | Low (<10¹) | Low (<10¹) |
| resistance | | | 10³) | 10²) | | |
| (Ω/sq) | | | | | | |
| Sheet | Low (2.98) | Low (3.24) | High (10²- | High (10³) | Open | Relatively |
| resistance | | | 10³) | | circuit at | high (10¹) |
| at 100% | | | | | 30% strain | |
| strain | | | | | | |
| (Ω/sq) | | | | | | |
| Withstand | Yes (>10⁴ | Yes (>10³ | Yes (>10³ | Yes (>10³ | Yes (>10³ | Yes (>10⁴ |
| cyclic | cycles) | cycles) | cycles) | cycles) | cycles) | cycles) |
| loading | | | | | | |
| Electro- | Insensitive | N/A | Sensitive | Sensitive | Relatively | Not |
| chemical | | (corrosion | | | sensitive | reported |
| strain | | limited) | | | | |
| sensitivity | | | | | | |
| (ΔZ/Zo at | | | | | | |
| 1 kHz) | | | | | | |
| Electro- | High | Low | High | High | High | High |
| chemical | | | | | | |
| stability | | | | | | |
| Electro- | Generalizable | Corrosion | Inefficient | Insufficient | Not | Strain |
| chemical | | limited | for | for | demonstrated | sensitive |
| functionality | | | stimulation | stimulation | | sensing |
| (stimulation/ | | | | | | |
| biomarker | | | | | | |
| sensing) | | | | | | |

The values from the above table can be derived from the present disclosure, as well as the following references and publications: D. Jung et al., Highly conductive and elastic nanomembrane for skin electronics. Science. 373, 1022-1026 (2021). doi: 10.1126/science.abh4357; Y. J. Yun et al., Highly Elastic Graphene-Based Electronics Toward Electronic Skin. Adv. Funct. Mater. 27 (2017), p. 1701513. doi: 10.1002/adfm.201701513; N. Liu et al., Ultratransparent and stretchable graphene electrodes. Sci Adv. 3, e1700159 (2017). 4869-8336-5267.1

doi: 10.1126/sciadv.1700159; Z. Yu, X. Niu, Z. Liu, Q. Pei, Intrinsically stretchable polymer light-emitting devices using carbon nanotube-polymer composite electrodes. Adv. Mater. 23, 3989-3994 (2011). doi: 10.1002/adma.201101986; L. Cai et al., Super-stretchable, transparent carbon nanotube-based capacitive strain sensors for human motion detection. Sci. Rep. 3, 3048 (2013). doi: 10.1038/srep03048; S. Lee et al., Ultrasoft electronics to monitor dynamically pulsing cardiomyocytes. Nat. Nanotechnol. 14, 156-160 (2019). doi: 10.1038/s41565-018-0331-8; C. F. Guo, T. Sun, Q. Liu, Z. Suo, Z. Ren, Highly stretchable and transparent nanomesh electrodes made by grain boundary lithography. Nat. Commun. 5, 3121 (2014). doi: 10.1038/ncomms4121; F. Xu, Y. Zhu, Highly conductive and stretchable silver nanowire conductors. Adv. Mater. 24, 5117-5122 (2012). doi: 10.1002/adma.201201886; Y. Chen, R. S. Carmichael, T. B. Carmichael, Patterned, Flexible, and Stretchable Silver Nanowire/Polymer Composite Films as Transparent Conductive Electrodes. ACS Appl. Mater. Interfaces. 11, 31210-31219 (2019). doi: 10.1021/acsami.9b11149; Y. Yu, et al., Ultra-stretchable conductors based on buckled super-aligned carbon nanotube films. Nanoscale. 7, 10178-10185 (2015). doi: 10.1039/c5nr01383f; Y. Wang et al., A highly stretchable, transparent, and conductive polymer. Sci Adv. 3, e1602076 (2017). doi: 10.1126/sciadv.1602076; H. He et al., Biocompatible Conductive Polymers with High Conductivity and High Stretchability. ACS Appl. Mater. Interfaces. 11, 26185-26193 (2019). doi: 10.1021/acsami.9b07325.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably coupleable,” to each other to achieve the desired functionality. Specific examples of operably coupleable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

Although the present embodiments have been particularly described with reference to preferred examples thereof, it should be readily apparent to those of ordinary skill in the art that changes and modifications in the form and details may be made without departing from the spirit and scope of the present disclosure. It is intended that the appended claims encompass such changes and modifications.

1. A device, comprising:
a strain-insensitive bioelectrode (SIB) having:
a first layer implemented by a tissue interfacing element comprised of a thin film;
a second layer implemented by an interconnection element; and
a third layer implemented by an adhesive and anisotropically conductive film (ACF) that integrates the tissue interfacing element and the interconnection element.

2. The device of claim 1, wherein the thin film comprises a material that is brittle but has high electrochemical performance.

3. The device of claim 1, wherein the thin film comprises gold.

4. The device of claim 1, wherein the thin film comprises platinum.

5. The device of claim 1, wherein the thin film comprises iridium oxide.

6. The device of claim 1, wherein the first layer exploits interfacial channel cracks as means of tensile strain energy release.

7. The device of claim 6, wherein a cracked fragment experiences minimal strain while remaining electrically connected to the system, preserving the overall active surface area of the SIB.

8. The device of claim 1, wherein the first, second and third layers of the SIB decouple the bioelectronic materials configuration into the tissue interfacing element for electron transfer and the interconnection element for electron transport.

9. The device of claim 1, where in the ACF comprises isolated conductive particles embedded in a stretchable matrix.

10. The device of claim 1, wherein interconnection element comprises intrinsically stretchable and highly conductive traces.

11. The device of claim 10, wherein the traces comprise silver nanowire-based traces.

12. The device of claim 10, wherein the traces are inlaid in a surface layer of a soft rubbery matrix.

13. The device of claim 12, wherein the soft rubbery matrix comprises polyurethane acrylate.

14. The device of claim 1, wherein the SIB has a thickness of about 140 μm.

15. The device of claim 1, wherein the SIB has a softness of about 10 MPa.

16. The device of claim 1, wherein the SIB has a stretchability of greater than 150%.

17. A method, comprising:
obtaining a strain-insensitive bioelectrode (SIB) by:
implementing a first layer by a tissue interfacing element comprised of a thin film;
implementing a second layer by an interconnection element; and
implementing a third layer by an adhesive and anisotropically conductive film (ACF) that integrates the tissue interfacing element and the interconnection element.

18. The method of claim 17, further comprising:
decoupling the bioelectronic materials configuration of the SIB into the tissue interfacing element for electron transfer and the interconnection element for electron transport.

19. The method of claim 17, further comprising:
exploiting and coupling surface channel cracks within the tissue interface element and anisotropic out-of-plane/in-plane electron conduction within the interconnection element to eliminate strain effects on device performance.

20. The method of claim 17, wherein the thin film comprises a material that is brittle but has high electrochemical performance.

21. The method of claim 17, wherein the thin film comprises one of gold, platinum, iridium oxide and carbon.
